(19) United States
(12) Patent Application Publication
(10) Pub. No.: US 2008/0215070 A1
(43) Pub. Date: Sep. 4, 2008

(54) System and Method for Tensioning a Suture

(76) Inventor: Philip L Gildenberg, Houston, TX (US)

Correspondence Address: Vinson & Elkins, First City Tower, 1001 Fannin Street, Suite 2500, Houston, TX, 77002-6760, US

(21) Appl. No.: 11/988,333

(22) PCT Filed: Nov. 30, 2005
(86) PCT No.: PCT/US2005/043154

§ 371 Date: Jul. 11, 2007

Related U.S. Application Data
(60) Provisional application No. 60/632,182, filed on Dec. 1, 2004.

(51) Int. Cl. A61B 17/04

(52) U.S. Cl. 606/148

(57) ABSTRACT

The present invention provides a novel forceps for use in suturing. The forceps include a pulley or pulley-like feature that provides an advantageous mechanism for putting tension on a suture. In certain embodiments, the pulley or pulley-like device can measure or set the tension applied to a suture. Use of the forceps is especially advantageous in endoscopic or robot assisted surgeries.

### REFERENCE TO RELATED APPLICATION

This application claims priority to U.S. Provisional application No. 60/632,182, filed Dec. 1, 2004.

### BACKGROUND OF THE INVENTION

Suturing is used in a variety of medical procedures. In simple terms, suturing includes joining two or more surfaces or edges together using a repetitive sewing type process. For example, a thread or similar type material, i.e., suture, may be used during a surgical procedure to close a wound or join tissue.

With regard to surgical procedures, there is a continuing emphasis in the medical profession to minimize the size of a surgical opening. This advantageously minimizes the stress on the patient's body and usually results in an expedited patient recovery time. Unfortunately, small surgical openings result in small surgical fields, thus complicating the process of suturing. In other words, small surgical fields limit the space a surgeon has for suturing.

Endoscopic surgery compromises access to the surgical field even further. In suturing, either with a conventional needle or one designed for endoscopic surgery, it is still necessary to pull the remainder of the suture length through the tissue and to pull on the suture sufficiently to apply the proper tension to the emerging suture. It is especially difficult to pull excess length through the tissue and attain proper tissue tension in robotic surgery, since the field is limited, manipulation of the instruments is done mechanically and may not include all the manipulations that can be applied to the instruments in conventional surgery, the technique is guided entirely by visual feedback, and there is not haptic feedback to guide tissue tensioning.

With a typical suture process, a needle is used to repeatedly feed a suture through the tissue that is to be joined together. For example, on the first pass through the tissue, the suture is tied to anchor it at the end of the incision to be approximated. Then the needle is pulled through the tissue to begin the continuous suturing. Once the needle is pulled through the tissue to be approximated, it is necessary to pull through the tissue the remaining length of suture, i.e., that length of suture attached to the needle but not yet pulled through the passage that was produced by the needle on its way through the tissue. Because of limited space, forceps are typically used to maneuver the needle, thread, and tissue. To pull the thread through using conventional techniques, it is necessary to remove the needle grasped with, for example, needle forceps or needle holder, from the surgical field or to grasp the suture repeatedly with another set of forceps (tissue forceps) in a hand-over-hand fashion.

Repeatedly withdrawing the needle from the surgical field is a particular problem in minimally invasive, endoscopic, or robotic surgery because, in such instances, the needle is removed from the surgeon's sight while it passes close to the surrounding tissue and may injure it. That risk is repeated when returning the needle to the surgical field of view. If the suture is particularly long, it may be difficult to control the intervening length of the suture or to adjust the traction accurately.

Ordinarily, needle forceps are used to push the needle through the tissue to be joined, and when the needle clears the tissue, tissue forceps are used to pull the suture taut. Forceps are not designed for grasping or otherwise interacting with the suture. With repeated grasping and releasing, the forceps can damage the suture. Moreover, it is sometimes necessary to remove the forceps from the visible surgical field making the technique awkward and putting surrounding structures at risk.

What is needed, therefore, is a system and method for providing an efficient means of pulling the suture through the tissue and accurately adjusting its tension. The present invention is directed to overcoming, or at least reducing the effects of, one or more of the problems set forth above.

### SUMMARY OF THE INVENTION

The invention consists of modifying existing tissue forceps to add a small pulley (a moveable attachment) or pulley-like device (an attachment that does not rotate, the upper surface of which is shaped like a pulley) near the end of one of the two arms. After the needle is pulled through the tissue during the continuous suturing process, the suture between the needle and the tissue from which the needle has emerged is set within the pully-like device, and as the tissue forceps are drawn away from the tissue the remaining suture is pulled through the tissue until the proper tension on the tissue closure is achieved. The tension maybe sustained by the suturing device as the needle is again passed through the tissue at the next point of suturing, and the process is repeated.

### BRIEF DESCRIPTION OF THE FIGURES

FIG. 1 depicts a forceps with an attached pulley-like system

### DETAILED DESCRIPTION OF SPECIFIC EMBODIMENTS

There are many types of forceps used in suturing. The size varies depending on the size of the needle, the thickness of the tissue, and the activity to be performed. The configuration of the jaws of the forceps varies depending on the characteristics of the tissue to be held and the preference of the surgeon. The present invention is applicable to any type of forceps, e.g., tissue forceps, needle forceps, etc., and, as will be described, results in a significant improvement to the forceps suture tying capabilities.

It is usually the case that two forceps are used during a suture procedure. One forceps, for example, may be used to hold the needle (needle forceps, needle holder, etc.), while the other forceps is used to hold the tissue (tissue forceps). During the procedure, the function of the forceps is ordinarily alternated. For example, the needle forceps may push the needle partially through the tissue on one side, and the tissue forceps may then grasp the needle on the other side. The needle forceps releases the needle and the tissue forceps pulls the needle the rest of the way through the tissue. Once the needle is pulled through, the tissue forceps may hand the needle off to the needle forceps, and the process is repeated as desired.

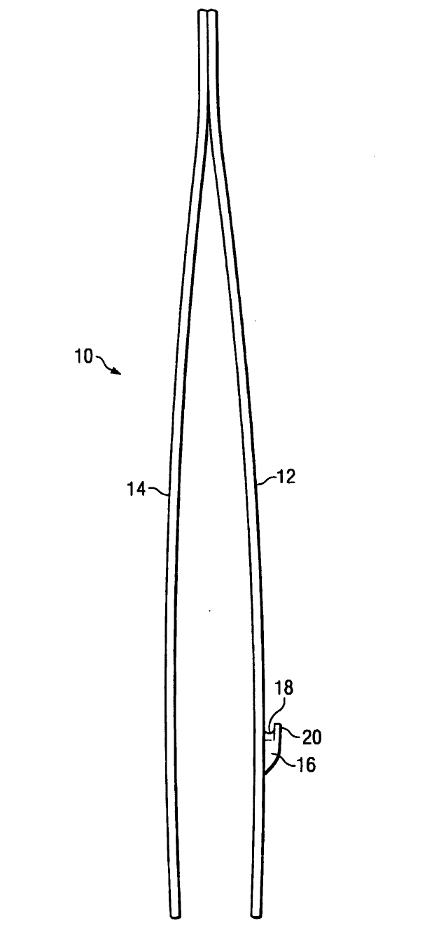

Referring to FIG. 1, illustrative forceps 10 having a first arm 12 and a second arm 14 are shown. As described, the forceps 10 may be used for grasping, manipulating, or extracting during a surgical procedure. A pulley-like device 16 is shown attached to an end of the first arm of the forceps. In this illustrative example, the pulley-like device 16 does not move, but offers a surface 18 to draw the suture through the tissue to a desired tension. The shape of the pulley 16 is configured to allow the suture easy travel without slipping from it. In this example, the pulley is shaped with a tab 20 that creates a slot for holding the suture.

The pulley-like system consists of a small knob that is made as part of any desired tissue forceps. The upper surface is shaped like one-half of a pulley to form a seat for the suture, and the lower surface is tapered to minimize the risk that it may catch on surrounding tissue. The pulley-like device may not rotate like a pulley, but may remain static. The pulley-like device could be part of either a conventional tissue forceps, a needle forceps or needle holder, or any instrument designed for endoscopic or robotic surgery.

“Pulley” and “pulley-like device” can be used interchangeably in the context of this document.

The pulley-like device 16 may be comprised of a metal, alloy, or any other substance. Moreover, the pulley 16 may be made part of the forceps 10 using any number of processes. In this case, the pulley 16 is comprised of surgical stainless steel, and it is attached to the forceps 10 using an adhesive or welding process. Alternatively, the pulley 16 may be included in the forceps manufacturing process. For example, the forceps 10 may be forged from one piece of stainless steel, and the shape of the pulley-like device may be added to the forceps casting form. In still other embodiments, the pulley-like device 16 may be added to the forceps as a snap-on component.

In another embodiment (not shown), the pulley includes a spring or other tensioning device, and the pulley is moveably attached to the forceps. For example, a slot may be provided in the first arm of the forceps, and a tensioning device, e.g., spring, incorporated therein. The pulley may be attached to the tensioning device, such that it becomes slideably operable along the slot when a certain amount of force is applied. Such a configuration may be used to assist a surgeon to accurately adjust the tension the suture places on the tissue. For example, as the suture is drawn across the pulley, movement by the pulley may be used to signal when a predetermined amount of force is reached. Such forceps may be designed for a specific torque or tension. Alternatively, the tensioning device may be adjustable, thus providing customizable movement of the pulley.

In one illustrative embodiment, forceps having a pulley or pulley-like device may be used by a surgeon in a confined surgical field to tie a suture. For convenience, forceps incorporating a pulley shall be referred to hereafter as pulley forceps. In a suture tying procedure, after the needle is passed through the tissue, the needle is held near the point it has just passed through the tissue using, for example, conventional forceps, e.g., needle forceps. It should be appreciated, however, that the needle might be held in place using pulley forceps. Advantageously, in this position, the needle should be in the surgical view of the surgeon.

The pulley or pulley-like device of the pulley forceps is placed under the length of suture between the needle and the tissue. In this position, the suture rests on the pulley. The pulley forceps is pulled toward the surgeon, with the needle held in place, for example, with the needle forceps. As the pulley forceps is pulled toward the surgeon, the suture is drawn across the pulley until it is brought to a desired tension. Using the pulley to snug the suture, it is only necessary to pull the forceps half as far out of the incision as if the tension were applied by withdrawing the needle forceps from the field. Once a desired tension is reached, the pulley forceps is returned to grasp the tissue for the next pass of the needle and suture. This process may be repeated as desired.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention.

What is claimed:

1. A forceps comprising:
a first arm;
a second arm attached to the first arm; and
a pulley or pulley-like device attached to the first arm or the second arm.

2. The forceps of claim 1, wherein the pulley or pulley-like device further comprises a surface for a suture to be drawn across providing tension on the suture; and a tab to secure the suture on the surface.

3. The forceps of claim 1, wherein the pulley or pulley-like device further comprises a tensioning device.

4. The forceps of claim 3, wherein the tensioning device is a spring.

5. The forceps of claim 3, wherein the pulley or pulley-like device is slideably attached to the forceps and the pulley location on the forceps indicates the tension being applied to a suture.

6. The forceps of claim 1, wherein the pulley or pulley-like device is a snap-on feature added to the forceps.

7. The forceps of claim 1, wherein the first arm, the second arm and the pulley or pulley-like device are cast as one unitary piece.

8. A modified forceps comprising:
a forceps; and
a pulley or pulley-like device attached to the forceps.

9. The forceps of claim 8, wherein the pulley or pulley-like device further comprises a surface for a suture to be drawn across providing tension on the suture; and a tab to secure the suture on the surface.

10. The forceps of claim 8, wherein the pulley or pulley-like device further comprises a tensioning device.

11. The forceps of claim 10, wherein the tensioning device is a spring or attached to a spring.

12. The forceps of claim 10, wherein the pulley or pulley-like device is slideably attached to the forceps and the pulley or pulley-like device location on the forceps indicates the tension being applied to a suture.

13. The forceps of claim 8, wherein the pulley or pulley-like device is a snap-on feature added to the forceps.

14. The forceps of claim 8 wherein the pulley or pulley-like device is included in the manufacture of the forceps.

15. A method for tying a suture comprising:
(a) passing a suture needle through a tissue to be sutured;
(b) grasping the suture needle with a grasping instrument;
(c) securing the suture on a pulley or pulley-like device connected to a second grasping instrument; and
(d) pulling the second grasping instrument distally away from the tissue to put tension on the suture.

16. The method of claim 15, wherein the pulley or pulley-like device further comprises a surface for a suture to be drawn across providing tension on the suture; and a tab to secure the suture on the surface.

17. The method of claim 15, wherein the pulley or pulley-like device further comprises a tensioning device.

18. The method of claim 17, wherein the tensioning device is a spring or device connected to a spring.

19. The method of claim 17, wherein the pulley or pulley-like device is slideably attached to the second grasping instrument and the pulley or pulley-like device location on the second grasping instrument indicates the tension being applied to a suture.

20. The method of claim 15, wherein the pulley or pulley-like device is a snap-on feature added to the second grasping instrument.

21. The method of claim 15, wherein the grasping instrument and the pulley or pulley-like device are cast as one unitary piece.
